(19) United States
(12) Patent Application Publication
(10) Pub. No.: US 2013/0253468 A1
(43) Pub. Date: Sep. 26, 2013

(54) Carrier for Releasing a Therapeutic Substance in Response to the Presence of an Enzyme

(71) Applicant: Advanced Cardiovascular Systems, Inc., Santa Clara, CA (US)

(72) Inventor: Syed F.A. Hossainy, Hayward, CA (US)

(73) Assignee: Advanced Cardiovascular Systems, Inc., Santa Clara, CA (US)

(21) Appl. No.: 13/896,173

(22) Filed: May 16, 2013

Related U.S. Application Data
(63) Continuation of application No. 11/227,732, filed on Sep. 14, 2005.
Application No. 11/227,732 is a continuation of application No. 10/099,427, filed on Mar. 15, 2002, now abandoned.

(51) Int. Cl. A61L 27/54; A61L 27/34

(52) U.S. Cl. CPC A61L 27/54; A61L 27/34
USPC 604/500; 604/264

(57) ABSTRACT

A stent with a coating including a composition that releases a therapeutic substance in response to an enzyme is disclosed. The composition includes a polymer or polypeptide that includes an amino acid sequence which is recognized and cleaved by at least one of the matrix metalloproteinases (MMP).

### CROSS-REFERENCE TO RELATED APPLICATIONS

This application is a continuation of U.S. application Ser. No. 11/227,732, filed on Sep. 14, 2005, and published on Jan. 12, 2006, as United States Patent application publication number 2006/0009840 A1, which is incorporated by reference herein in its entirety, including any drawings, and U.S. application Ser. No. 11/227,732 is a continuation of U.S. application Ser. No. 10/099,427, filed on Mar. 15, 2002.

### BACKGROUND OF THE INVENTION

1. Field of the Invention

This invention relates to a biocompatible carrier containing a therapeutic substance for introducing the substance to a certain target cell population, such as smooth muscle cells or inflammatory cells, requiring modulation to ameliorate a diseased state. Moreover particularly, the invention is directed to a composition that releases a therapeutic substance in response to an enzyme.

2. Description of the Background

In order to provide an efficacious concentration of a medication to a diseased or treatment site, systemic administration of the medication often produces adverse or toxic side effects for the patient. Local delivery is a preferred method of treatment in that smaller total levels of medication are administered in comparison to systemic dosages, but are concentrated at a specific site. Local delivery thus produces fewer side effects and achieves more favorable results. The use of polymeric carriers is a commonly employed method of local drug delivery. Biocompatible polymers, such as polyvinyl acetate, ethylene vinyl alcohol, and polyureathanes, to name a few, can be used as microparticles or coating for implantable devices, such as stents, for the sustained release of the drug.

There are several factors that determine the release rate of a drug from a polymer carrier. The ability of the polymer to absorb water is one factor. If the matrix readily absorbs water, the drug is released quickly. Solubility of the drug in the medium is another factor. If the drug is readily soluble in the medium, then the drug release rate will be quick. Other factors include the microphase state of the drug in the polymer and the ratio of the drug to the polymer. Whether the microphase state of the drug is crystalline or amorphous or whether the active ingredient is phase dispersed, are also other considerations. Although there is a plurality of factors which can be modified to customize the in vitro release rate of a drug, there is also a need to be able to control the release kinetics based on the activity of the biological environment in which the carrier is placed.

Accordingly, it is desired to provide a carrier that specifically releases a drug in response to the biological activity of the environment in which the carrier is placed, such as in response to the endogenously occurring protease.

### SUMMARY

In accordance with one embodiment of the invention, a composition for introducing a therapeutic compound to a mammal, such as in a blood vessel of a mammal is provided. The composition comprises the therapeutic compound in a polymeric carrier including a matrix metalloproteinase (MMP) cleavable polypeptide. The matrix metalloproteinase (MMP) cleavable polypeptide can be, for example, collagen, elastin, fibronectin, laminin, proteoglycan, tropoelastin, silk elastin, gelatin or combinations thereof. The matrix metalloproteinase (MMP) can be MMP-1, MMP-2, MMP-3, MMP-7, MMP-8, MMP-9, MMP-10, or MMP-11. In one embodiment, the therapeutic compound can be for reducing or eliminating restenosis or the development thereof. In yet another embodiment, the therapeutic compound can be an inhibitor of matrix metalloproteinase.

In accordance with another embodiment, a stent is provided comprising a radially expandable body and a therapeutic substance carried by the stent for the release of the therapeutic substance in response to the exposure of the stent to an enzyme, such as a metalloproteinase enzyme. The therapeutic substance can be carried by a coating on the stent, the coating comprising a matrix metalloproteinase cleavable peptide. Alternatively, the therapeutic substance can be carried by a polymeric coating on the stent, the polymeric coating including bonds that are cleavable by a matrix metalloproteinase enzyme. The therapeutic substance can be a matrix metalloproteinase inhibitor, can be for reducing, delaying or eliminating migration and proliferation of vascular smooth muscle cells, or can be for the treatment of restenosis.

In accordance with another embodiment of the invention, a method of forming a coating for a prosthesis, such as a stent, is provided. The method comprises applying a composition comprising a therapeutic compound to the prosthesis such that the coating is configured to release the therapeutic compound in response to exposure to the enzyme.

In accordance with yet another embodiment, a method of inhibiting restenosis is provided, comprising depositing into a designated region of the blood vessel a carrier containing a therapeutic substance, wherein the carrier is degradable when exposed to an enzyme in the blood vessel.

### DETAILED DESCRIPTION

As used herein, “matrix metalloproteinases” (MMP's) are a class of extracellular enzymes including collagenase, stromelysin, and gelatinase which are believed to be involved in tissue destruction which accompanies a large number of disease states varying from arthritis to cancer. This group of enzymes with different substrate specificity contributes to the degradation of extracellular matrix comprising such complex components as collagen, proteoglycan, elastin, fibronectin, and laminin. In particular, MMP cleavage of the ECM protein facilitates cellular invasion and migration.

MMPs include interstitial collagenase (MMP-1), 72 kDa gelatinase (also known as type IV collagenase or gelatinase A; MMP-2), 92 kDa gelatinase (also known as type IV collagenase or gelatinase B; MMP-9), stromelysin-1 (MMP-3), matrilysin (MMP-7), neutrophil collagenase (MMP-8), stromelysin-2 (MMP10) and stromelysin-3 (MMP-11). With the exception of MMP-7, the primary structure among the family of reported MMPs comprises essentially an N-terminal propeptide domain, a Zn⁺⁺ binding catalytic domain and a C-terminal hemopexin-like domain. In MMP-7 there is no hemopexin-like domain. MMP-2 and MMP-9 contain an additional gelatin-binding domain. In addition, a proline-rich domain highly homologous to a type V collagen alpha 2 chain is inserted in MMP-9 between the Zn⁺⁺ binding catalytic domain and the C-terminal hemopexin-like domain.

The propeptides of MMPs generally consist of approximately 80-90 amino acids containing a cysteine residue, which interacts with the catalytic zinc atom via its side chain thiol group. A highly conserved sequence ( . . . ProGlyCysGlyXaaProAsp (SEQ ID NO:1) . . . ) is usually present in the propeptide. Removal of the propeptide by proteolysis results in zymogen activation, as all members of the MMP family are produced in a latent form. The catalytic domain contains two zinc ions and at least one calcium ion coordinated to various residues.

The structural determinants within these enzymes which confer the ability to degrade various substrates appear to be localized within discrete domains. For example, the ability of the collagenases to degrade triple-helical collagen requires the presence of the C-terminal hemopexin-like domain. In contrast, stromelysins degrade a variety of substrates in a manner which is independent of the C-terminal hemopexin-like domain. Unique to the 72 kDa and 92 kDa gelatinases is an additional domain composed of three fibronectin type II repeats inserted in tandem within the zinc-binding catalytic domain. This fibronectin-like domain is required for the gelatinases to bind efficiently to type I gelatin and type IV collagen. Matrilysin is the simplest member of this family of enzymes in that it contains only a zymogen domain and a catalytic zinc-binding domain.

The interactions of cells with the extracellular matrix are important for the normal development and function of the organism. Modulation of cell-matrix interactions occurs through the action of unique proteolytic systems responsible for hydrolysis of a variety of the extracellular matrix components. By regulating the integrity and composition of the extracellular matrix structure, these enzyme systems play a pivotal role in the control of signals elicited by matrix molecules, which regulate cell proliferation, differentiation, and cell death. The turnover and remodeling of the extracellular matrix must be highly regulated since uncontrolled proteolysis contributes to abnormal development and to the generation of many pathological conditions characterized by either excessive degradation or a lack of degradation of extracellular matrix components.

MMPs play a role in normal and pathological processes, including embryogenesis, wound healing, inflammation, restenosis, arthritis, apoptosis and cancer. In highly metastatic tumor cells, there are reports of conspicuous expression of type IV collagenase (MMP-2, MMP-9) which mainly degrade type IV collagen (Cancer Res., 46:1-7, 1986; Biochem. Biophys. Res. Commun., 154:832-838, 1988; Cancer, 71:1368-1383, 1993). In a sense, the degree of matrix metalloproteinase expression correlates with the degree of cancer malignancy. The association of MMPs with cancer metastasis has raised considerable interest because they represent an attractive target for development of novel antimetastatic drugs aimed at inhibiting MMP activity.

The metastasis of tumor cells progresses via destruction of basement membranes, invasion into and effusion from blood vessels, successful implantation on secondary organs, further growth and the like. The extracellular matrix that blocks tumor metastasis is composed of various complex components, including type IV collagen, proteoglycans, elastin, fibronectin, laminin, heparan sulfate, and the like. And these matrix metalloproteinases, with their distinct substrate specificities are responsible for the degradation of the extracellular matrix. Among these MMPs, it has been reported that type IV collagenase (MMP-2 and MMP-9) is highly expressed in high metastatic tumor cells. Type IV collagen is a principal constituent of basement membranes. The regulation of MMP activation is believed to be performed in steps including at least transcription level, a step for converting a proenzyme form wherein its enzymatic activity is latent into an active enzyme form, and controls by tissue inhibitor of metalloproteinase (TIMP) being a specific inhibitor against MMPs, and the like.

As used herein, “MMP cleavable polypeptide” refers to that polypeptide having an amino acid sequence, which is recognized and cleaved by at least one of the MMPs. The recognition may be specific to a particular MMP or it may be general to all MMPs. An example of a polypeptide that is cleaved by an MMP is collagen, gelatin, elastin and silk-elastin. However, it is understood that the polypeptide sequence need not be a full-length protein such as collagen or elastin. It is understood that any fragment or derivative of these MMP substrates that is cleaved by an MMP falls within the purview of the invention. Furthermore, it is understood that the MMP cleavable polypeptide encompasses polypeptides that are at least partly synthetic and/or chimeric, such as silk-elastin. It is also understood that the MMP cleavable polypeptide may include those polypeptides that may not be completely cleaved by MMP. For example, an MMP cleavable polypeptide may be linked to a non-MMP cleavable polypeptide.

As used herein, “elastin-collagen matrix combination (ECM)” refers to an exemplified type of polypeptide combination that is a proteolytic substrate for an MMP. ECM is combined with an active agent, such that when the ECM/active agent combination composition is placed in the environment of the expressed MMP, the ECM polypeptide is degraded and the active agent is released at the local site. The ECM/active agent combination may be further combined with other polymeric carriers. It is understood that elastin and collagen are not limited to the intact protein. Rather, they include fragments and other derivatives, so long as they are cleavable by an MMP to release the active agent. It is also understood that depending on the level of desirable sustained release rate of the active agent, the amino acid sequence of the polypeptide matrix may be varied so that the polypeptide contains a desired number of cleavage sites. The presence of a cleavage site can be determined by using well known biochemical procedures, such as by using labeled oligopeptides as substrates to determine the proteolytic ability of various MMPs in order to ascertain the amino acid sequences that may be incorporated into an MMP cleavable polypeptide.

As used herein, “MMP inhibitor” refers to any chemical compound that is effective in inhibiting the biological activity of matrix metalloproteinases such as collagenase, stromelysin, gelatinase and elastase. Numerous compounds are known to be matrix metalloproteinase inhibitors, and any such inhibitor compound can be utilized in the practice of the embodiments of this invention.

Thus, MMP inhibitor compounds are useful for the treatment of gelatinase-, stromelysin-, collagenase-, TNF alpha-, MT-MMP-1 and 2-, and macrophage metalloelastase-dependent pathological conditions in mammals. Such conditions include malignant and non-malignant tumors by inhibiting tumor growth, tumor metastasis, tumor progression or invasion and/or tumor angiogenesis, including, for example, breast, lung, bladder, colon, ovarian and skin cancer. Other conditions to be treated with the compounds of the invention include rheumatoid arthritis, osteoarthritis, bronchial disorders (such as asthma by inhibiting the degradation of elastin), atherosclerotic conditions (by e.g. inhibiting rupture of atherosclerotic plaques), as well as acute coronary syndrome, heart attacks (cardiac ischemia), strokes (cerebral ischemias), restenosis and stenosis after angioplasty, and vascular ulcerations, ectasia and aneurysms.

As used herein, “analogs” or “derivatives” refers to any variation of a therapeutic compound, active ingredient or drug, terms which are used interchangeably, that retains the biological activity and/or functionality of the agent. As these terms are used in relation to a polypeptide such as collagen, elastin or silk-elastin in a matrix that is combined with an active ingredient or any polypeptide which MMP cleaves, the analog or derivative refers to the polypeptide that may be fragmented or mutated, but is still cleaved by an MMP and yet retains at least some of the functional characteristics of the protein.

As used herein, “biologically derived carrier molecule” refers to molecules that are naturally found in a mammal, which is also useful as a carrier. Examples of it include albumin, glycosaminoglycans, hyaluronic acid, and the like. These biologically derived carrier molecules may be blended along with MMP cleavable polymeric carriers.

As used herein, “early stage cancer” refers to the early aspects of cancer progression, such as local invasion and micrometastasis.

As used herein, “inhibiting” cellular activity means reducing, delaying or eliminating smooth muscle cell hyperplasia, restenosis, vascular occlusions, platelet activation, or inflammatory response, particularly following biologically or mechanically mediated vascular injury or trauma or under conditions that would predispose a mammal to suffer such a vascular injury or trauma. The invention is also directed to treating or inhibiting early stage cancer. The effects of reducing, delaying, or eliminating neoplastic proliferation may be determined by methods known to one of ordinary skill in the art, including, but not limited to, angiography, ultrasonic evaluation, fluoroscopy imaging, fiber optic visualization, or biopsy and histology. Biologically mediated vascular injury includes, but is not limited to injury caused by or attributed to autoimmune disorders, alloimmune related disorders, infectious disorders including endotoxins and herpes viruses such as cytomegalovirus, metabolic disorders such as atherosclerosis, and vascular injury resulting from hypothermia, irradiation and cancer. Mechanical mediated vascular injury includes, but is not limited to vascular injury caused by catheterization procedures or vascular scraping procedures such as percutaneous transluminal coronary angioplasty, vascular surgery, stent placement, transplantation surgery, laser treatment, and other invasive procedures which disrupted the integrity of the vascular intima or endothelium. The active ingredient of the invention is not restricted in use for therapy following vascular injury or trauma; rather, the usefulness of the active ingredient will also be determined by the ingredient's ability to inhibit cellular activity of smooth muscle cells or inhibit various diseases including restenosis and early stage cancer.

As used herein, “neoplastic proliferation” means new and abnormal growth or proliferation of tissue, which may be benign or cancerous.

As used herein, “proliferation” of smooth muscle cells means increase in cell number.

As used herein, “smooth muscle cells” include those cells derived from the medial and adventitia layers of the vessel which proliferate in intimal hyperplastic vascular sites following vascular trauma or injury. Under light microscopic examination, characteristics of smooth muscle cells include a histological morphology of a spindle shape with an oblong nucleus located centrally in the cell with nucleoli present and myofibrils in the sarcoplasm. Under electron microscopic examination, smooth muscle cells have long slender mitochondria in the juxtanuclear sarcoplasm, a few tubular elements of granular endoplasmic reticulum, and numerous clusters of free ribosomes. A small Golgi complex may also be located near one pole of the nucleus.

As used herein, “abnormal” or “inappropriate” proliferation means division, growth or migration of cells occurring more rapidly or to a significantly greater extent than which typically occurs in a normally functioning cell of the same type, i.e., hyperproliferation.

During the process of neoplastic proliferation of smooth muscle cells, matrix metalloproteinase or matrix metalloprotease (MMP) is generated in the area to assist in the proliferation of the neoplastic cells. The expression of the metalloproteinase may be used to degrade a polypeptide matrix or backbone in a polymeric carrier in order to release the active ingredient that is deposited within the carrier.

Composition

The composition of the invention may comprise a MMP cleavable polymer or polypeptide, or a polymer containing the polypeptide with or without various other types of biologically derived carrier molecules such as albumin, hyaluronic acid, glycosaminoglycan, and the like. The composition may comprise the blending of various polymers and copolymers, including polyethylene glycol with other biologically derived carrier molecules. A combination of both blending and conjugation may be employed for the purposes of this invention. It is understood that any suitable non-cleavable polymeric carrier may be used in the practice of the invention so long as the active ingredient and MPP cleavable polypeptide are included in the composition.

In one embodiment of the invention, drug delivery can be achieved by incorporating a therapeutic substance into a cross-linked elastin-collagen matrix combination (ECM). The ECM coated drugs may be dispersed in a polymer coating or can be applied as is on the stent. In accordance with another embodiment, labile chemical bond that is cleavable by MMP can be incorporated into a polymer backbone, wherein the polymer is loaded with a therapeutic substance. During the burst of MMP production in the blood vessel, ECM and/or the polymer is degraded by the endogenous MMP protease and the therapeutic substance is released into the environment.

Active Ingredient

The active ingredient may be variously referred to as a drug, therapeutic agent, bioactive agent, therapeutic compound, or therapeutically active compound. The active ingredient may be a large molecule, or a drug with low water solubility, which is blended with the MMP cleavable carrier. Such large molecules may include polypeptides such as antibodies, enzymes, and non-enzymatic proteins. The active ingredient may also inhibit the proliferative activity of vascular smooth muscle cells. In particular, the active ingredient can be aimed at inhibiting abnormal or inappropriate migration and/or proliferation of smooth muscle cells. In one embodiment, the active ingredient may inhibit matrix metalloproteinase activity to treat conditions such as cancer, early stage of cancer, or restenosis.

Representative examples include antiproliferative substances such as actinomycin D, or derivatives and analogs thereof (manufactured by Sigma-Aldrich 1001 West Saint Paul Avenue, Milwaukee, Wis. 53233; or COSMEGEN available from Merck). Synonyms of actinomycin D include dactinomycin, actinomycin IV, actinomycin I₁, actinomycin X₁, and actinomycin C₁. The active agent can also fall under the genus of antineoplastic, antiinflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL® by Bristol-Myers Squibb Co., Stamford, Conn.), docetaxel (e.g. Taxotere®, from Aventis S.A., Frankfurt, Germany) methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.) Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein platelet membrane receptor antagonist antibody, recombinant hirudin, and thrombin inhibitors such as Angiomax™ (Biogen, Inc., Cambridge, Mass.) Examples of such cytostatic or antiproliferative agents include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate include alpha-interferon, genetically engineered epithelial cells, rapamycin and dexamethasone.

The dosage or concentration of the active ingredient required to produce a favorable therapeutic effect should be less than the level at which the active ingredient produces toxic effects and greater than the level at which non-therapeutic results are obtained. The dosage or concentration of the active ingredient required to inhibit the desired cellular activity of the vascular region can depend upon factors such as the particular circumstances of the patient; the nature of the trauma; the nature of the therapy desired; the time over which the ingredient administered resides at the vascular site; and if other therapeutic agents are employed, the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

Prosthesis

Should the composition be used with a prosthetic device, the prosthesis can be, for example, a self-expandable stent, a balloon-expandable stent, a pattern stent such as microdepot stent, a stent-graft or a graft. Also included are coronary shunts, anasmatosis devices, valves and other implantable medical devices. The underlying structure of the prosthesis can be virtually any design. The prosthesis can be made of a metallic material or an alloy such as stainless steel. The prosthesis can also be made from a bioabsorbable or biostable polymer.

The following examples are offered by way of illustration and not by way of limitation.

### EXAMPLES

### Example 1

Water-in-oil emulsion/solvent evaporation technique: Tropoelastin/elastin and collagen combination (Sigma) is dissolved in deionized water at pH 4.0 at 10% w/v (5% tropoelastin, 5% collagen). Liquid paraffin (100 ml) with 1% w/v of Span 80 is placed in a 400 ml beaker, and agitated at 400 rpm with a 3-bladed propeller stirrer (diameter 5 cm), linked to a stirring motor (Tecmatic SD2). 1 gram of actinomycin (Ac-D) is dispersed in 20 ml of the ECM solution. The mixture is then poured into the paraffin, and evaporation of the water proceeds for 24 hours at 50° C. The microspheres are collected in Buchner filter, washed in 50 ml of ether, and allowed to dry at room temperature for 24 hours. 10% w/v ECM-coated Ac-D microspheres are spray coated from a pentane formulation or a Techspray formulation (Techspray, Inc., Amarillo, Tx) on the stent that is primed with an ethylene vinyl alcohol copolymer (EVAL). The total amount on the stent is 200 micrograms. A 2% EVAL in dimethylacetamide (DMAC) was sprayed on the elastin-collagen matrix combination (ECM)-Ac-D coated stent as a top coating for a total amount of 300 micrograms.

### Example 2

In situ desolvation technique: Tropoelastin/elastin and collagen combination is dissolved in deionized water at pH 4.0 at 2% w/v (1% tropoelastin, 1% collagen). The solution is spray coated on the stent for a total deposition of 200 micrograms. 10% w/w solution of Ac-D is made in tetrahydrofuran (THF). The ECM coated stent is immersed in this solution and gradually deionized water is added into the solution. The Ac-D phase separates and precipitates on the stent. After 2 minutes of deposition, the stent is taken out and dried at room temperature for 6 hours in a convection oven. ECM-Ac-D coated stents are coated with the 2% ECM solution for a deposition of 200 micrograms. The ECM-sandwiched Ac-D is further coated with 2% EVAL in DMAC as a top coating for a deposition of 100 micrograms.

### Example 3

Tropoelastin/elastin and collagen combination is dissolved in deionized water at pH 4.0 at 2% w/v (1% tropoelastin, 1% collagen). The solution is spray coated on the stent for a total deposition of 200 micrograms. 10% w/w solution of Ac-D is made in THF. The ECM coated stent is immersed in this solution, and gradually deionized water is added into the solution. The Ac-D phase separates and precipitates on the stent. After 2 minutes of deposition, the stent is taken out and dried in room temperature for 6 hours in a convection oven. ECM-Ac-D coated stents are coated with the 2% ECM solution for a deposition of 200 micrograms. The ECM-sandwiched Ac-D is further coated with 3% EVAL-poly-n-butyl methacrylate (PBMA) (1% EVAL: 2% PBMA, 33% cyclohexanone, 64% DMAC) as a top coating for a deposition of 300 micrograms.

### Example 4

An ethylene vinyl alcohol solution is made by dissolving 10 grams of EVAL in 90 grams of DMAC. ECM coated Ac-D microparticles are suspended in the EVAL solution by mixing 20 grams of the microparticles with 80 grams of EVAL solution. Microparticles are selected within a size range of 0.5 to 2 microns in the characteristic length. The final suspension is constantly stirred to prevent flocculation. The stents are dipped in the final suspension and centrifuged at 6000 rpm for 60 seconds for a smooth defect-free coating.

### Example 5

Stents that are coated as in Example 4 are subsequently spray-coated with a 3% EVAL-PBMA (1% EVAL, 2% PBMA, 33% cyclohexanone, 64% DMAC) as a top coating for a deposition of 300 micrograms.

### Example 6

Coating of Silk Elastin: 5% aqueous solution of silk elastin is mixed with a 2% by weight suspension of beta estradiol. The solution is spray coated on the stent.

While particular embodiments of the present invention have been shown and described, it will be obvious to those skilled in the art that changes and modifications can be made without departing from this invention in its broader aspects and, therefore, the appended claims are to encompass within their scope all such changes and modifications as fall within the true spirit and scope of this invention.

What is claimed is:

1. A stent comprising:
a radially expandable body having a surface;
a coating over the surface comprising
a therapeutic agent, and
a polymer modified by incorporation of chemical bonds cleavable by matrix metalloproteinase (MMP) into the polymer backbone, a MMP cleavable polypeptide linked to a non-MMP cleavable polypeptide, an MMP cleavable polypeptide that is at least partially synthetic, or a combination thereof;
wherein an MMP cleavable polypeptide is a polypeptide with an amino acid sequence which is recognized and cleaved by at least one of the matrix metalloproteinases.

2. The stent of claim 1, wherein the coating further comprises a matrix metalloproteinase inhibitor dispersed in the polymer coating.

3. The stent of claim 1, wherein the therapeutic substance is capable of reducing, delaying or eliminating migration and/or proliferation of vascular smooth muscle cells.

4. The stent of claim 1, wherein the therapeutic substance is capable of reducing, delaying or eliminating restenosis.

5. The stent of claim 1, where the coating comprises a polymer modified by incorporation of chemical bonds cleavable by MMP into the polymer backbone.

6. The stent of claim 1, where the coating comprises an MMP cleavable polypeptide linked to a non-MMP cleavable polypeptide.

7. The stent of claim 1, where the coating comprises an MMP cleavable polypeptide that is at least partially synthetic.

8. The stent of claim 1, where the therapeutic agent is selected from the group consisting of actinomycin D, paclitaxel, docetaxel, methotrexate, azathioprine, vincristine, vinblastine, fluorouracil, doxorubicin hydrochloride, mitomycin, sodium heparin, low molecular weight heparins, heparinoids, hirudin, argatroban, forskolin, vapiprost, prostacyclin, dextran, D-phe-pro-arg-chloromethylketone (synthetic antithrombin), dipyridamole, recombinant hirudin, bivalirudin, angiopeptin, captopril, cilazapril, lisinopril, nifedipine, lovastin, nitroprusside, phosphodiesterase inhibitor, prostaglandin inhibitor, thioprotease inhibitor, triazolopyrimidine, nitric oxide, pemirolast potassium, colchicine, rapamycin, dexamethasone, and combinations thereof.

9. The stent of claim 5, where the therapeutic agent is selected from the group consisting of actinomycin D, paclitaxel, docetaxel, methotrexate, azathioprine, vincristine, vinblastine, fluorouracil, doxorubicin hydrochloride, mitomycin, sodium heparin, low molecular weight heparins, heparinoids, hirudin, argatroban, forskolin, vapiprost, prostacyclin, dextran, D-phe-pro-arg-chloromethylketone (synthetic antithrombin), dipyridamole, recombinant hirudin, bivalirudin, angiopeptin, captopril, cilazapril, lisinopril, nifedipine, lovastin, nitroprusside, phosphodiesterase inhibitor, prostaglandin inhibitor, thioprotease inhibitor, triazolopyrimidine, nitric oxide, pemirolast potassium, colchicine, rapamycin, dexamethasone, and combinations thereof.

10. The stent of claim 6, where the therapeutic agent is selected from the group consisting of actinomycin D, paclitaxel, docetaxel, methotrexate, azathioprine, vincristine, vinblastine, fluorouracil, doxorubicin hydrochloride, mitomycin, sodium heparin, low molecular weight heparins, heparinoids, hirudin, argatroban, forskolin, vapiprost, prostacyclin, dextran, D-phe-pro-arg-chloromethylketone (synthetic antithrombin), dipyridamole, recombinant hirudin, bivalirudin, angiopeptin, captopril, cilazapril, lisinopril, nifedipine, lovastin, nitroprusside, phosphodiesterase inhibitor, prostaglandin inhibitor, thioprotease inhibitor, triazolopyrimidine, nitric oxide, pemirolast potassium, colchicine, rapamycin, dexamethasone, and combinations thereof.

11. The stent of claim 7, where the therapeutic agent is selected from the group consisting of actinomycin D, paclitaxel, docetaxel, methotrexate, azathioprine, vincristine, vinblastine, fluorouracil, doxorubicin hydrochloride, mitomycin, sodium heparin, low molecular weight heparins, heparinoids, hirudin, argatroban, forskolin, vapiprost, prostacyclin, dextran, D-phe-pro-arg-chloromethylketone (synthetic antithrombin), dipyridamole, recombinant hirudin, bivalirudin, angiopeptin, captopril, cilazapril, lisinopril, nifedipine, lovastin, nitroprusside, phosphodiesterase inhibitor, prostaglandin inhibitor, thioprotease inhibitor, triazolopyrimidine, nitric oxide, pemirolast potassium, colchicine, rapamycin, dexamethasone, and combinations thereof.

12. A method of treating or preventing restenosis comprising delivering to a site in a patient's vasculature where restenosis is occurring or is anticipated to occur, a stent according to claim 1.
